(19) United States
(12) Patent Application Publication
(10) Pub. No.: US 2020/0402060 A1
(43) Pub. Date: Dec. 24, 2020

(54) MEDICAL BENEFIT FRAUD PROTECTION SYSTEM AND METHOD

(71) Applicant: Zero Copay Program, Inc., New Hyde Park, NY (US)

(72) Inventor: Yuriy Davydov, Old Brookville, NY (US)

(73) Assignee: Zero Copay Program, Inc., New Hyde Park, NY (US)

(21) Appl. No.: 16/448,559

(22) Filed: Jun. 21, 2019

(51) Int. Cl. G06Q 20/40; G06Q 40/08; G06F 21/31

(52) U.S. Cl. CPC G06Q 20/4016; G16H 40/00; G06F 21/31; G06Q 40/08

(57) ABSTRACT

Medical benefit fraud protection system and method are disclosed herein. The method, in an embodiment, performs the following steps: receiving a provisioning request from a first processor controlled by a product related entity, wherein the provisioning request comprises a benefit specification from the product related entity, including limitations of use; configuring a benefit processing business logic of the benefit processing system with the provisioning request; receiving a benefit submission from a second processor controlled by a dispensary entity; determining an approval decision or a denial decision of the benefit submission based on the limitations of use in the benefit specification; and processing or denying the benefit submission based on such determination.

### BACKGROUND

Conventional benefit management systems typically are based on monolithic software that incorporates business logic, database management, configuration, and administration in a system having a single administrative domain of access. In such systems, operators, typically employed by a single entity, control the system to make modifications, run reports, and add services and features.

The typical workflow in these conventional systems involves human intervention to fulfill work orders or prepare reports for the various business entities that are involved in the operation of the system. For example, the on-ramping process for a new entity (e.g., a medical manufacturer) might include discussions about how that entity's business logic should interact with benefits and orders placed in the system, leading to the design and implementation of such logic, often in the form of configuration files. These files are then loaded into the system during a maintenance window or during another specified time of day with the goal of beginning operation in a future time period.

In another example, periodic reports are conventionally prepared for the various business entities by running database reporting software local to the systems. These reports are then sent to the different entities for review. Any additional reports are conventionally filled by work orders taken by the operators of the business management system, and then forwarded to the requesting entity.

In a further example, loading of new benefits programs conventionally requires an operator of the benefit management system to manually and laboriously work with the sponsor of the new benefits programs to design the business logic, and then reconfigure the system, typically during a maintenance window.

Furthermore, the known system fails to sufficiently safeguard against fraud and abuse in the the processing and determination of benefits by different entities.

### SUMMARY

In one embodiment, presented herein is a medical benefit fraud protection system. The system includes one or more data storage devices storing a plurality of instructions configured to be executed to perform the following steps. Receive a provisioning request from a first processor controlled by a product related entity. The provisioning request comprises specifications of a benefit specification from the product related entity. The medical benefit specification comprises a plurality of limitations of use associated with the product related entity's policy or benefit rules. Configure a benefit processing business logic of the benefit processing system with the provisioning request. The configuring occurs during execution. Receive a benefit submission from a second processor controlled by a medical merchant or dispensary entity. The benefit submission relating to the benefit specification from the product related entity. Determine an approval decision or a denial decision of the benefit submission based on the limitations of use in the benefit specification. Upon determining an approval decision of the benefit submission, process the benefit submission using the benefit processing business logic based on the benefit specification. Upon determining a denial decision of the benefit submission, deny the benefit submission. Generate a benefit transaction record or a denial record related to the benefit submission. Display a first access interface to the product related entity and a second access interface to the dispensing entity. The displaying occurs during execution. The first and second administrative interfaces are accessible during execution and comprise different views to different portions of the benefit transaction record related to the benefit submission.

In another embodiment, a method includes the following. Receiving a provisioning request from a first processor controlled by a product related entity. The provisioning request comprises specifications of a benefit specification from the product related entity. The benefit specification comprise limitations of use. Configuring a benefit processing business logic of the benefit processing system with the configuring request. The configuring occurs during execution. Receiving a benefit submission from a second processor controlled by a medical merchant or dispensary entity. The benefit submission relating to the benefit specification from the product related entity. Determining an approval decision or a denial decision of the benefit submission based on the limitations of use in the benefit specification. Upon determining an approval decision of the benefit submission, processing the benefit submission using the benefit processing business logic based on the benefit specification. Upon determining a denial decision of the benefit submission, denying the benefit submission. Generating a benefit transaction record or a denial record related to the benefit submission. Displaying a first access interface to the product related entity and a second access interface to the dispensing entity. The displaying occurs during execution. The first and second administrative interfaces are accessible during execution and comprise different views to different portions of the benefit transaction record related to the benefit submission.

The above embodiments are exemplary only. Other embodiments are within the scope of the disclosed subject matter.

### BRIEF DESCRIPTION OF THE DRAWINGS

So that the manner in which the features of the disclosure can be understood, a detailed description may be had by reference to certain embodiments, some of which are illustrated in the accompanying drawings. It is to be noted, however, that the drawings illustrate only certain embodiments and are therefore not to be considered limiting of its scope, for the scope of the disclosed subject matter encompasses other embodiments as well. The drawings are not necessarily to scale, emphasis generally being placed upon illustrating the features of certain embodiments. In the drawings, like numerals are used to indicate like parts throughout the various views.

FIG. 1A is a block diagram of a medical benefit fraud protection or management system interoperating with other components or systems, in accordance with one or more aspects set forth herein.

FIG. 1B is a block diagram depicting further details of a medical benefit fraud protection or management system, in accordance with one or more aspects set forth herein.

FIG. 2A is a flowchart depicting a medical benefit fraud protection or management method, in accordance with one or more aspects set forth herein.

FIG. 2B is a flowchart depicting a medical benefit fraud protection or management method, in accordance with one or more aspects set forth herein.

FIGS. 3A-3C are flowcharts depicting a medical benefit fraud protection method, in accordance with one or more aspects set forth herein.

FIGS. 4-5 are flowcharts depicting business logic for execution by the benefit management system of FIG. 1A, in accordance with one or more aspects set forth herein.

FIG. 6 is a block diagram of a computer system, such as that employed by the benefit management system of FIG. 1A.

Corresponding reference characters indicate corresponding parts throughout several views. The examples set out herein illustrate several embodiments, but should not be construed as limiting in scope in any manner.

### DETAILED DESCRIPTION

The present disclosure relates to systems for protecting against medical benefit fraud and managing and processing benefit payments, and more particularly to a medical benefit fraud protection system that allows real time provisioning with respect to medical benefit offerings related to products. Depending on the embodiment, the medical benefit offerings can include coupons, discounts, rebates (instant or post-sale), freebies or other financial or monetary consideration.

In an embodiment, the medical benefit fraud protection system is usable by product related entities or manufacturers. At the same time, the medical benefit fraud protection system is usable by medical merchants who sell, resell, gift or otherwise provide the manufacturers' products to customers. The customers can include recipients, patients and agents acting on behalf of patients or people in need of medical products. By way of overview, these techniques represent practical applications that advance the field of benefit fraud protection and management, by: (a) increasing the efficiency of provisioning and handling of benefit submissions; and (b) including one or more security approval checkpoints based on benefit limitations or limitations of use established by the manufacturers. As one advantage, because the system continues to operate to handle benefit submissions and allow client interface access during provisioning of new business logic, users of the system can make real-time changes or additions to benefit specifications that are immediately reflected. In addition, real-time interface access, segmented by the accessing user (e.g., medical merchant, dispensary, product related entities or manufacturers), allows for different entities to gain different views into the same data, depending upon their role in the business logic and affiliated relationships.

Depending on the embodiment, a medical merchant can be a dispensary entity, pharmacy, medical clinic, medical equipment retailer, medical product dispensing company, retailer, store or other entity capable of selling, reselling, gifting or otherwise supplying medical products to customers. Depending on the transaction, the products may be goods or services that are manufactured, distributed, supplied, performed, rendered or marketed by the manufacturer. In some examples, a benefit offering may relate to a product that is or includes a physical item or good, such as a pharmaceutical product, drug, medical equipment, wheelchair, glucose meter, etc. In other examples, a benefit offering may relate to a product that is or includes a service or activity, such as a healthcare service, a home health visit service, a physical therapy session, etc. It should be understood that, if the product is or includes a service, the manufacturer of such product would be the conductor or performer of such product.

As described below, the medical benefit fraud protection system is operable to protect against or reduce fraud. It should be understood that the fraud can include any intentional wrongdoing of a person or other entity. For example, a customer may engaged in fraud by wrongfully or deceitfully obtaining a medical benefit that, according to the applicable manufacturer's policy or terms, is not to be provided to such customer.

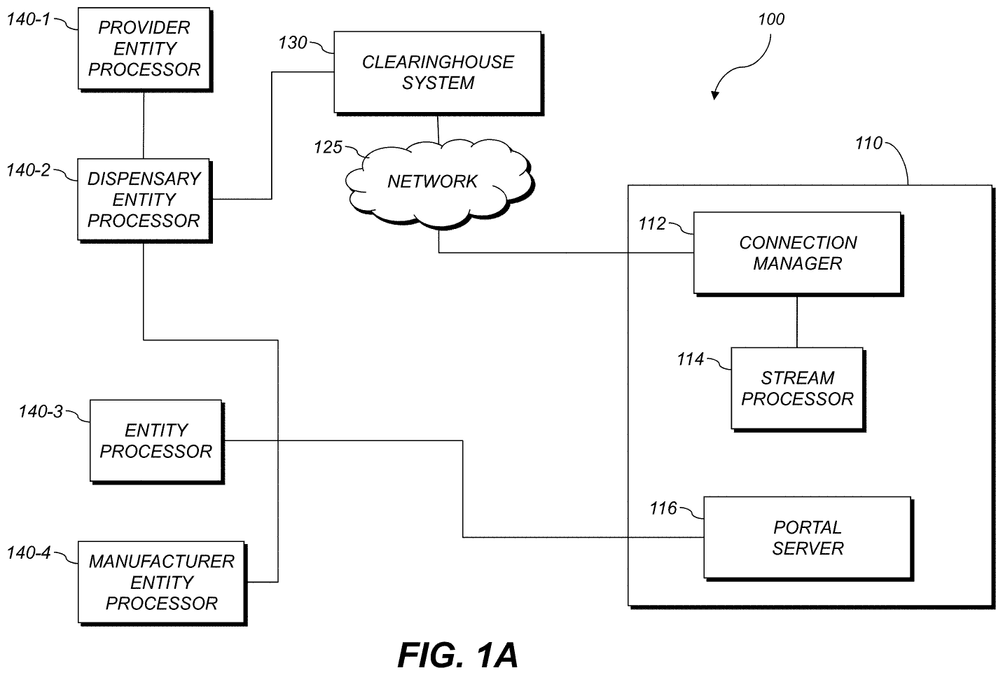

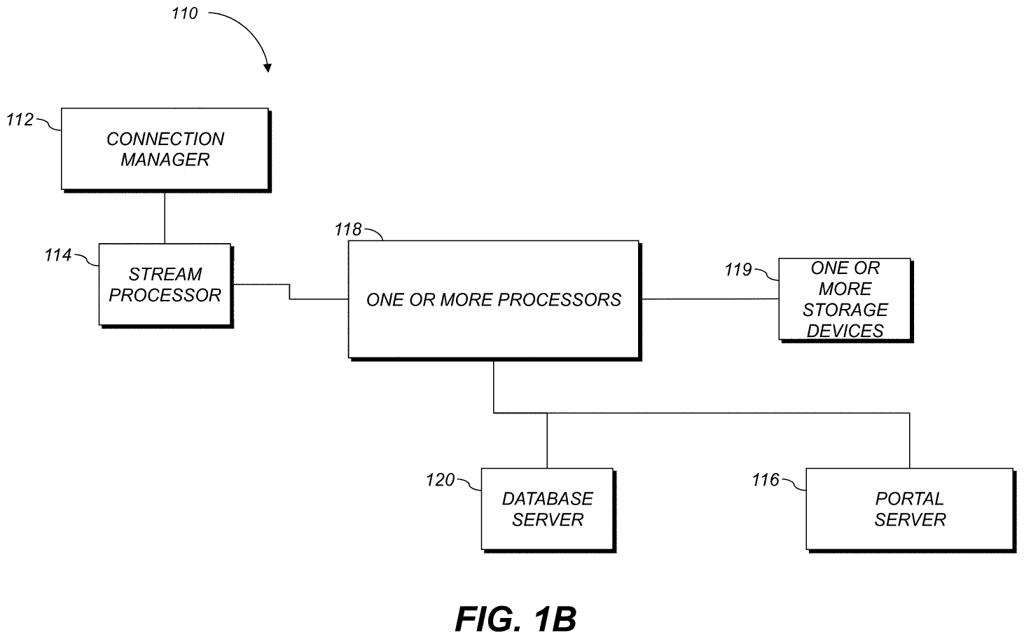

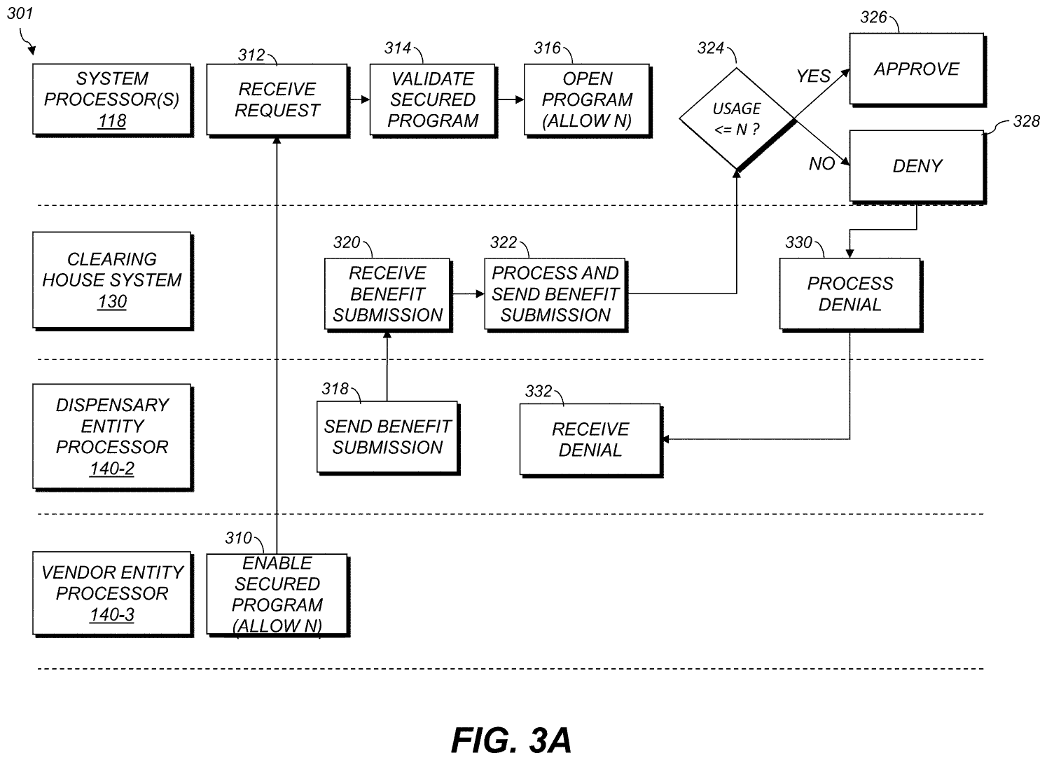

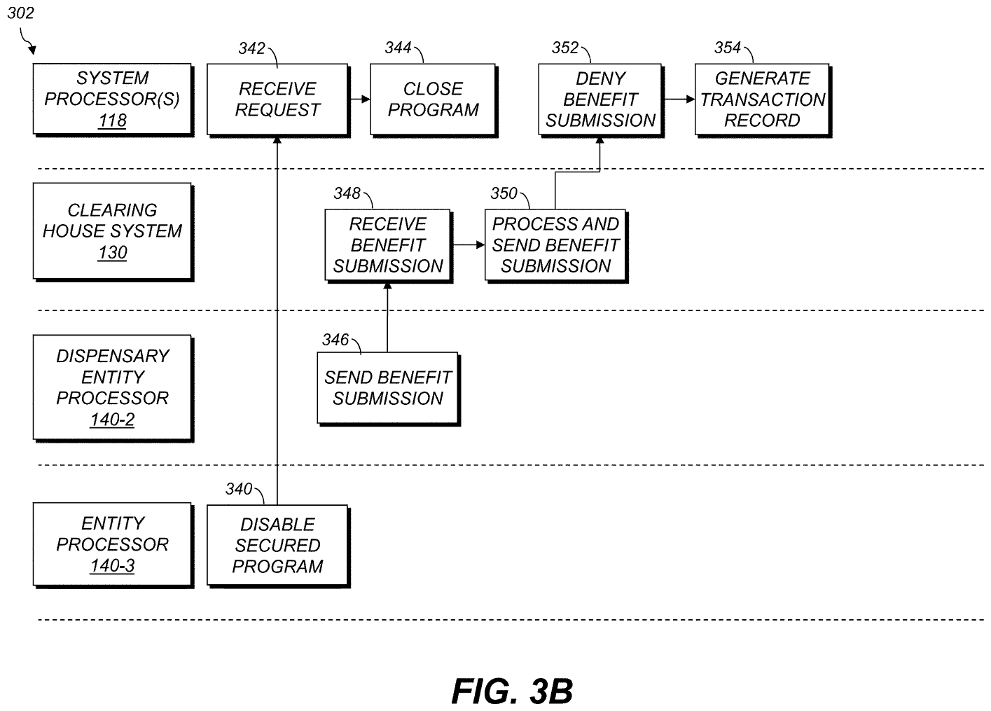

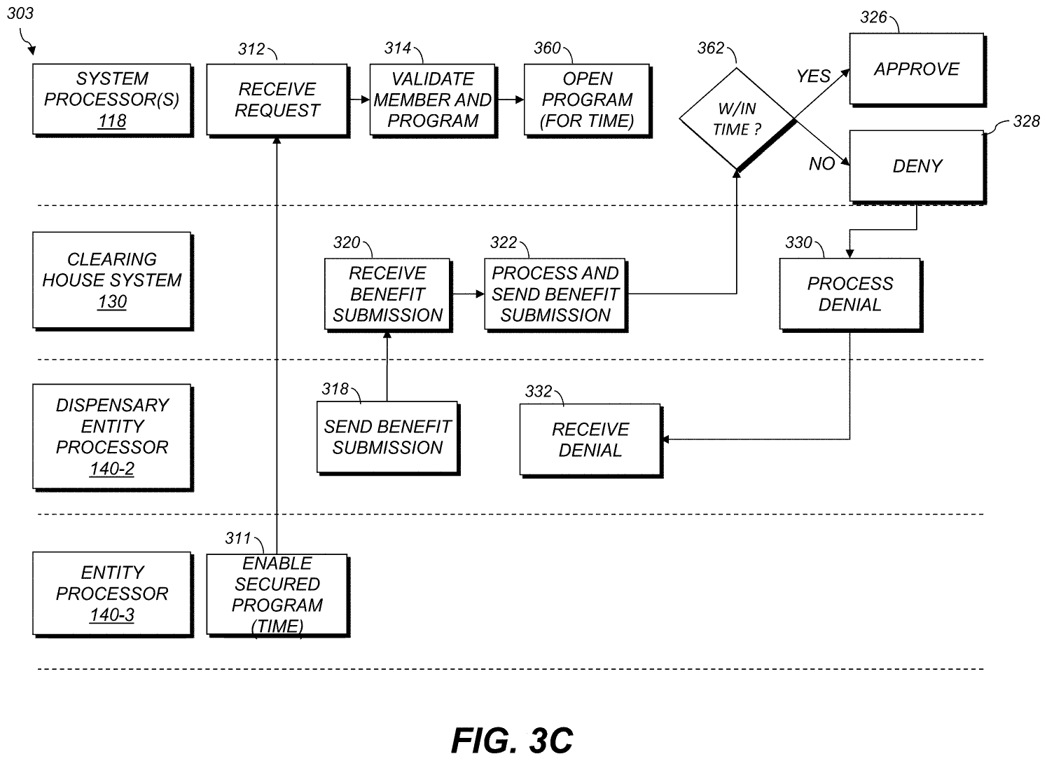

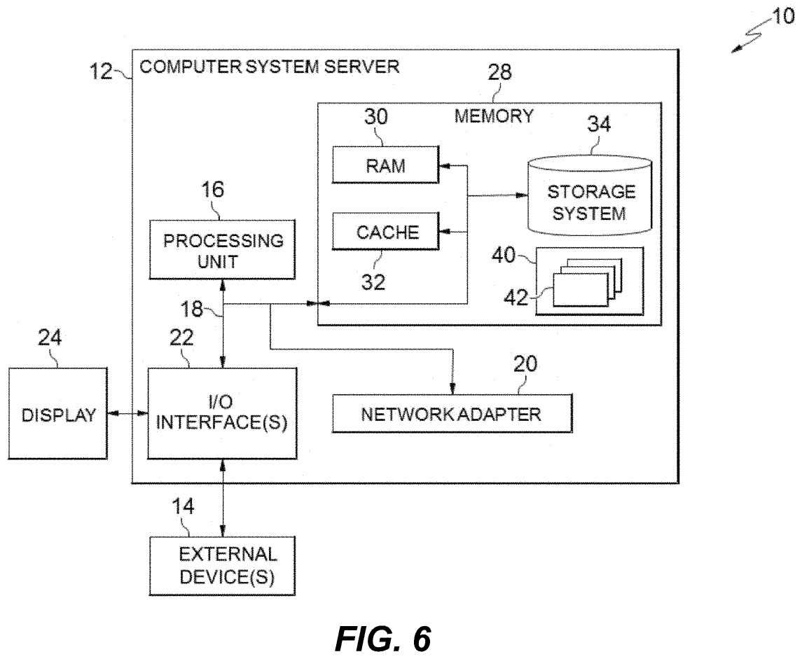

In the discussion that follows, FIGS. 1A-2B, FIGS. 4&5 and FIG. 6 relate details of the medical benefit fraud protection or management system and method, and FIGS. 3A-3C relate details of the medical benefit fraud protection method.

FIG. 1A is a block diagram of a benefit management system 110, interoperating with other components. In the embodiment of FIG. 1A, the benefit management system 110 includes a connection manager 112, a stream processor 114, and interface or portal server 116. For example, the connection manager 112, the stream processor 114, and the interface server 116 may execute on one or more processors, on one or more computer systems, depending on the capacity required of the system.

For instance, the connection manager 112 is responsible for conducting networking communications with various entities connected to the benefit management system 110 via a network 125. These entities include a clearinghouse 130, and processors of different entities, such as a provider entity processor 140-1, a medical merchant or dispensary entity processor 140-2, a vendor entity processor 140-3, or a manufacturer entity processor 140-4. Different consumers, or patients, typical work with providers, such as doctors or the like, to receive a prescription or other indication for a need for a product to be procured from a dispensary. Vendors and manufacturers are examples of product related entities who are involved in supplying a benefit for a product. Examples of product related entities include drug manufacturers, representatives or vendors of drug, medical equipment, and the like, non-profit organizations for facilitating lower patient costs, insurance companies, etc. In short, product related entities can include any entity that is involved in the supply chain of a product.

There may be multiple of these entities managed in the operation of the benefit management system 110. For example, the benefit management system 110 may support multiple dispensaries, such as pharmacies, medical drug dispensaries, medical equipment suppliers, or any other place where products that are potential the subject of a benefit are sold or supplied to consumers. Similarly, the benefit management system 110 may be support multiple product related entities that are involved in the provisioning of a benefit related to a product. As another example, a single clearinghouse or multiple clearinghouses may be used to allow the benefit management system 110 to receive claims submissions from multiple dispensaries.

In addition, the benefit management system 110 includes a stream processor 114 for scalable management of streams of data sent and received from the benefit management system 110 to and from the various entities. In one implementation, Apache Kafka, provided by the Apache Software Foundation of Maryland, USA, may be used as a stream processor.

Further, the benefit management system 110 includes an interface server 116, which can allow different views of the data to be presented to different entities, each of which may log into the interface server 116 with appropriate credentials. In one implementation, Apache Server, provided by the Apache Software Foundation of Maryland, USA, may be used as an interface server 116.

The components depicted in FIG. 1A may communicate via one or more networks 125. The network 125 may be a public or private network, such as any network based on the Internet Protocol, or any similar protocol.

FIG. 1B is a block diagram depicting further details of a benefit management system 110. Further implementation details of the benefit management system 110, in the embodiment of FIG. 1B, include one or more processors 118, one or more storage devices 119 and a database server 120. For example, the one or more processors 118 may be used to execute a plurality of program instructions stored on the one or more storage devices 119. In addition, the database server 120 may be used to store the provisioning requests, claim submissions, business logic, processed claims, configuration files, etc., as described below. In one implementation, the database server 120 may be a PostgresSQL database, available as an open source software from The PostgresSQL Global Development Group. In other implementation, any database, such as an SQL or object oriented database, may be used.

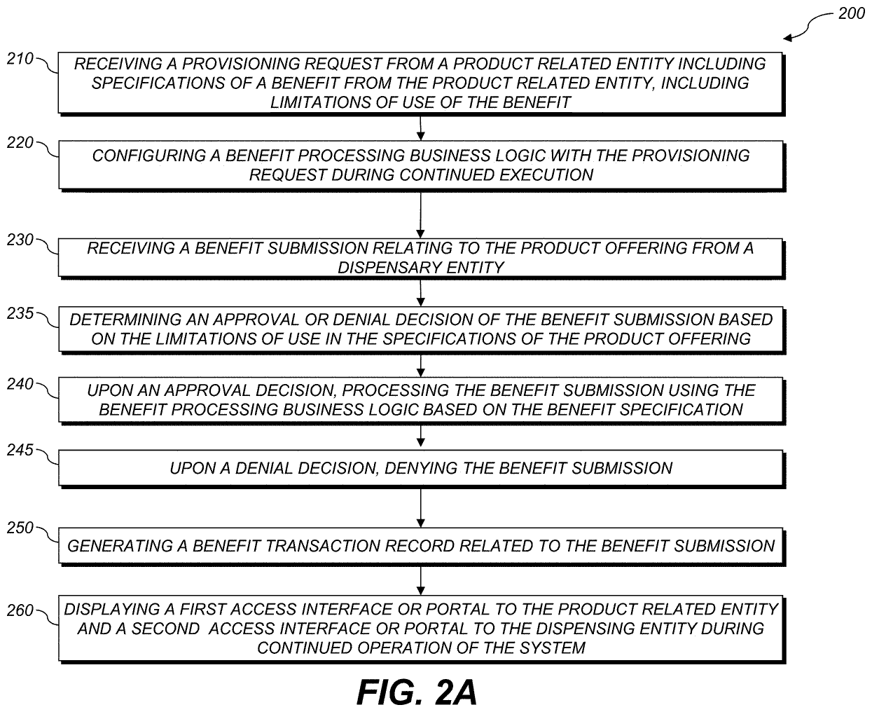

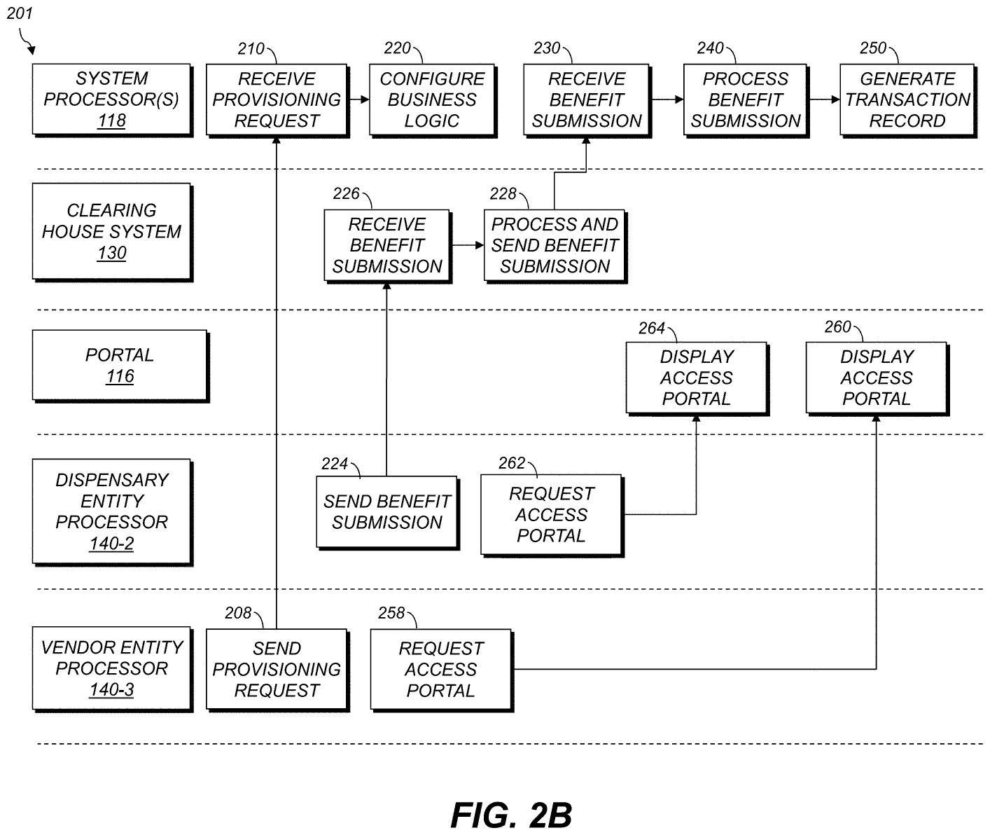

FIG. 2A is a flowchart depicting a medical benefit fraud protection or management method 200. In one embodiment, the method 200 at block 210 receives a provisioning request from a first processor controlled by a product related entity. The provisioning request includes specifications of a benefit specification from the product related entity.

In one example, the benefit management method may also receive one or more configuration requests from the product related entity or the dispensary entity, the configuration request comprising entity configuration information. In another example, a plurality of provisioning requests may be received from a plurality of product related entities, and may correspondingly include specifications of a plurality of benefit specifications from the plurality of product related entities.

The method 200 at block 220 provisions a benefit processing business logic of the benefit processing system with the provisioning request. Provisioning the business logic occurs during execution. For example, provisioning the benefit processing business logic may include generating one or more business rules based on the provisioning request.

In one example, the benefit management method provisions the benefit processing business logic of the benefit management system with the plurality of provisioning requests. In such a case, the provisioning may also occur during continued operation of the benefit management system.

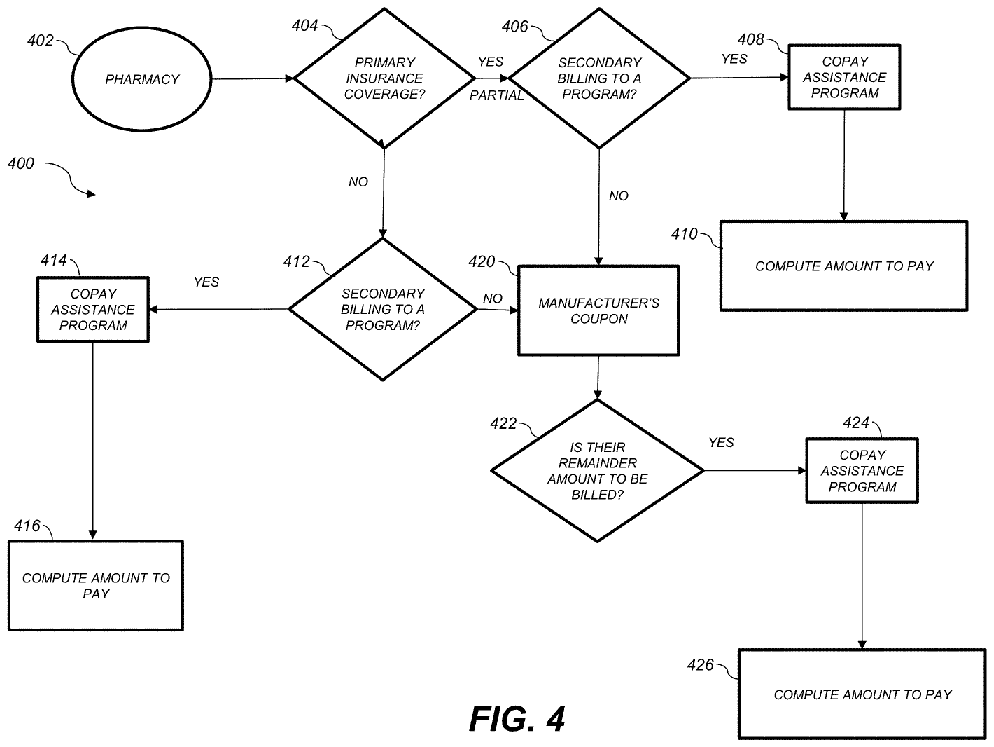

By way of explanation, the business logic may include rules that are triggered during the benefit processing specific to any of the business entities. The business rules can be updated in real-time by the benefit processing system to a specific entity without impacting the rules for any other entities. Examples of business logic are set forth in FIGS. 4 & 5.

The method 200 at block 230 receives a benefit submission from a second processor controlled by a dispensary entity. The benefit submission relating to the benefit specification from the product related entity.

In one example, the benefit submission is received via a clearinghouse. Different benefit submissions from different dispensaries may be received from the same or from different clearinghouses. In another example, the method securely validates the benefit submission before processing the benefit submission. In a further example, the benefit submission is not received via the clearinghouse, but instead is received directly by the benefit management method and system. In another example, a plurality of benefit submissions is received from a plurality of dispensary entities, each relating to a plurality of benefit specifications from the plurality of product related entities, and when received, the method generates a plurality of benefit transaction records related to the plurality of benefit submissions. Different submission types include those that are received from a clearinghouse or those received directly from an authorized dispensary.

Next, the method 200 at block 235 determines an approval decision or a denial decision of the benefit submission based on the limitations of use in the benefit specification. The limitations of use are associated with the product related entity's policy or benefit rules. For instance, the product related entity may have authorized this particular benefit only for a limited number of N uses by a specific patient, or for a limitation in the amount of time, or both. For example, the benefit may only be available to a particular user three times within a 6 month window. In another example, the limitations of use may deny the provision of the medical benefit to a pre-identified, blacklisted patient or customer. The requirement of such approval decision or denial decision constitutes a security checkpoint to enhance the security of the benefit processing.

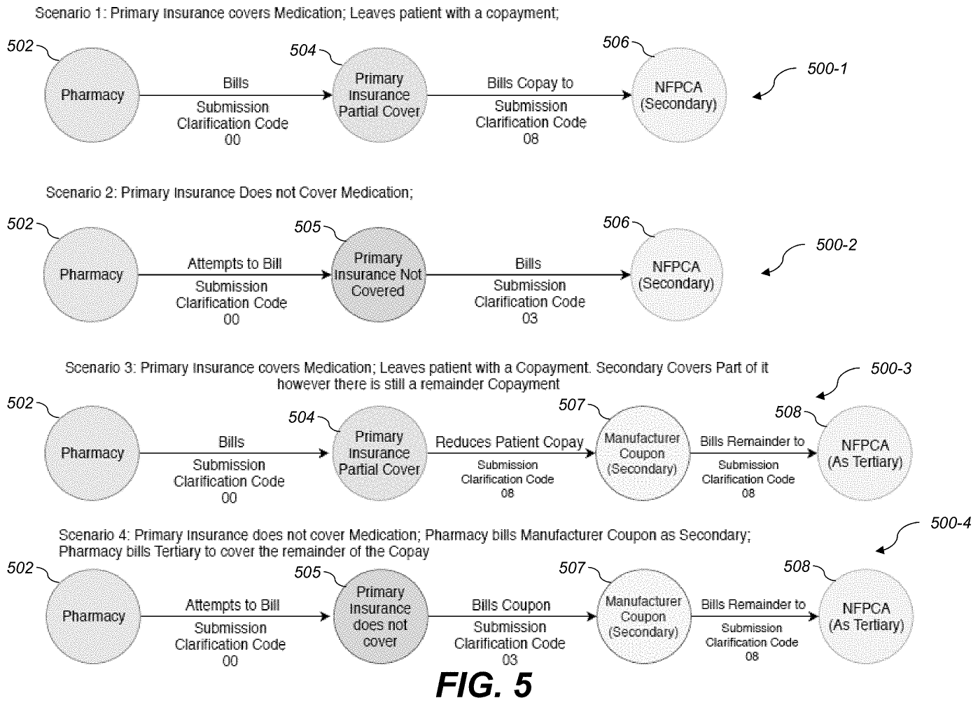

The method 200 at block 240, upon the approval decision, processes the benefit submission using the benefit processing business logic based on the benefit specification. For instance, a rules engine executes algorithms for each entity and their corresponding benefit submission, as depicted in FIGS. 4-5. However, the method 200 at block 245, upon a denial decision, denies the benefit submission.

Next, the method 200 at block 250 generates a benefit transaction record related to the benefit submission. In one example, the records received from dispensaries are rows of data that are processed during the transmission from the clearinghouse and are distributed into several segmented databases tables. The data is processed for efficiency and quick lookups that become available for each entity to view and consume their information.

At any time during the execution of any of the steps of the method 200, the method 200 at block 260 can display multiple interfaces for each entity. Each of the interfaces may comprise different views to different portions of the benefit transaction record related to the benefit submission. For example, each entity can be provisioned to view the data that is related to the entity itself. That data is trimmed based on access and can be viewed differently based on configured dashboard and different views of the interface.

In another example, the interface server can provide several different views for each of the different entities, to different portions of the plurality of benefit transaction records related to the plurality of benefit submissions.

FIG. 2B is a flowchart depicting a benefit management method 201. In the embodiment of FIG. 2B, the method 201 is performed by processor(s) 118 of the benefit management system 110 of FIG. 1A including the interface 116 thereof, the clearinghouse system 130, and two example entities. The two example entities for the embodiment of FIG. 2B include the dispensary entity processor 140-2 and the vendor entity processor 140-3. FIG. 2B shows 5 horizontal lanes with a component on the left and method steps performed by that component, in an embodiment, indicated its right in the same horizontal lanes. However, in different embodiments, the different steps of the method may be performed by other components, such as in a situation where multiple roles are performed by a single computer system.

Continuing now with FIG. 2B, the vendor entity processor 140-3 performs the step of the method 201 at block 208 to send a provisioning request to the system processor(s) 118. Next, the system processor(s) 118 performs the steps of the method 201 at block 210 to receive the provisioning request from the vendor entity processor 140-3. Next, the system processor(s) 118 performs the steps of the method 201 at block 220 to provision a benefit processing business logic of the benefit processing system 110 (FIG. 1A) with the provisioning request.

In one embodiment, the dispensary entity processor 140-2 performs the step of the method 201 at block 224 to send a benefit submission to the clearinghouse system 130. Then, the clearinghouse system 130 performs the steps of the method 201 at block 226 to receive the benefit submission from the dispensary entity processor 140-2. Next, the clearinghouse system 130 performs the steps of the method 201 at block 228 to process the benefit submission and send it to the appropriate entity, which in this case is the system processor(s) 118.

Continuing, the system processor(s) 118 performs the steps of the method 201 at block 230 to receive the benefit submission from the clearinghouse system 130 (which had ultimately received the benefit submission from the dispensary entity processor 140-2). Thereafter, the system processor(s) 118 performs the steps of the method 201 at block 240 to process the benefit submission using the benefit processing business logic based on the benefit specification. This could be the benefit processing logic previously provisioned at block 220, which may have been invoked repeatedly to load numerous different business processing logic and rules for all the different benefit programs supported by the specific instance of a benefit management system 110.

Next, the system processor(s) 118 performs the steps of the method 201 at block 250 generates a benefit transaction record related to the benefit submission. The record is stored internally in a database as discussed above, and memorializes the benefit transaction.

A person of ordinary skill in the art would readily understand that the steps discussed in the paragraphs above would be repeated hundreds, thousands, or millions of times, in the normal processing of a variety of benefit claims for numerous patients. The repeated transaction records would then be available in the benefit management system for subsequent access and interrogation.

For instance, any of the entities can then access the information as follows. In one example, the vendor entity processor 140-3 performs the steps of the method 201 at block 258 to request access to a client specific interface. The interface 116 then performs the steps of the method 201 at block 260 to display only an interface that is specific to the vendor entity processor 140-3, based on the permissions used for logging into the interface. Similarly, the dispensary entity processor 140-2 may perform the steps of the method at block 262 to request access to an interface, and the interface 116, in turn, may perform the steps of the method 201 at block 264 to display a specific access interface for the dispensary entity processor 140-2. These interface views will be different for each entity, and provide different views into the same data.

FIGS. 3A-3C are flowcharts depicting a medical benefit fraud protection method, in accordance with one or more aspects set forth herein. As depicted in FIG. 3A, the vendor entity processor 140-3 performs the step of a method 301 at block 310 to send a request to enable a secured program to the system processor(s) 118. Next, the system processor(s) 118 performs the steps of the method 301 at block 312 to receive the request from the vendor entity processor 140-3 to enable a new secured program. Next, the system processor(s) 118 performs the steps of the method 301 at block 314 to validate the new secured program, and thereafter, and block 316 opens the new secured program. For instance, the new secured program may allow one specific member to make N orders of a particular product, such as a pharmaceutical product.

By way of example, a vendor related entity, such as the manufacturer of a pharmaceutical, may decide to activate a copay assistance program. However, the entity may desire to control exactly who is allowed to make use of the program, and not allow other parties to make use of the program without authorization. In such a case, the vendor entity processor 140-3 will provide specific information when enabling the new secured program. For example, a specific patient or set of patients may be approved for a specific drug or family of drugs, for a specific number of refills, for a specific duration of time, with a specific dollar value of copay assistance. In one embodiment, the program will be completely disabled for everyone other than patients specifically authorized in the request sent at block 310 as noted above.

Next, the method 301 at block 318 sends a benefit submission to the clearinghouse system 130. Then, the clearinghouse system 130 performs the steps of the method 301 at block 320 to receive the benefit submission from the dispensary entity processor 140-2. Next, the clearinghouse system 130 performs the steps of the method 301 at block 322 to process the benefit submission and send it to the appropriate entity, which in this case is the system processor(s) 118. Continuing, the system processor(s) 118 performs the steps of the method 301 at block 324 to receive the benefit submission from the clearinghouse system 130 (which had ultimately received the benefit submission from the dispensary entity processor 140-2), and perform a check to see if the benefit submission meets the criteria of the secured program previously opened at block 316. If the criteria matches the secured program, the system processor(s) 118 performs the steps of the method 301 at block 326 to approve the benefit submission. The benefit may then be processed using the appropriate benefit processing business logic based on the benefit specification, as described previously. For example, meeting the criteria of the program may be defined by being a patient that was approved for a specific drug or family of drugs for, say, N times. Each time the prescription is filled, a specific counter may be incremented to track the usage of the program, and the program will be closed when N times are reached, in this example.

If the criteria does not match the secured program, in order to prevent, safeguard against, inhibit or protection against fraud, the system processor(s) 118 performs the steps of the method 301 at block 328 to deny the benefit submission. In such a case, the clearinghouse system 130 performs the steps of the method 301 at block 330 to process the denial, and the dispensary entity processor 140-2 processes the steps of the method 301 at block 332 to receive the denial. The denials and approvals can be viewed in the entity specific interfaces as described above.

In another example, a prescription may be canceled or reversed. In such a case, the business logic can be programmed to allow another prescription for the same patient and drug or family of drugs to replace the cancelled prescription. This could be achieved by decrementing the counter so that the program described above now has an extra usage count for use by the patient. In another example, a flag may be set indicating that the program is reopened for a specific duration of time to allow a reorder to process.

Next, FIG. 3B describes how a secured program can be disabled within the system. For instance, the vendor entity processor 140-3 performs the step of a method 301 at block 340 to send a request to disable the secured program to the system processor(s) 118. Next, the system processor(s) 118 performs the steps of the method 301 at block 342 to receive the request from the vendor entity processor 140-3 to disable or close the new secured program. Next, the system processor(s) 118 performs the steps of the method 301 at block 344 to close the secured program.

The fraud protection system will subsequently deny any attempt to make use of the closed program, as follows. For instance, the method 301 at block 346 sends a benefit submission to the clearinghouse system 130. Then, the clearinghouse system 130 performs the steps of the method 301 at block 348 to receive the benefit submission from the dispensary entity processor 140-2. Next, the clearinghouse system 130 performs the steps of the method 301 at block 350 to process the benefit submission and send it to the appropriate entity, which in this case is the system processor(s) 118. Continuing, the system processor(s) 118 performs the steps of the method 301 at block 352 to deny the benefit submission from the clearinghouse system 130 because the program had been closed previously at block 344, and generates an appropriate transaction to record the denial at block 354.

FIG. 3C depicts another example of a medical benefit fraud protection method, which is time based. As depicted in FIG. 3C, the vendor entity processor 140-3 performs the step of a method 301 at block 311 to send a request to enable a secured program to the system processor(s) 118. Next, the system processor(s) 118 performs the steps of the method 301 at block 312 to receive the request from the vendor entity processor 140-3 to enable a new secured program for a specific duration. Next, the system processor(s) 118 performs the steps of the method 301 at block 314 to validate the new secured program, and thereafter, and block 360 opens the new secured program. For instance, the new secured program may allow one specific member to make orders of a product during a particular calendar window, e.g., a certain number of months.

Next, the method 301 at block 318 sends a benefit submission to the clearinghouse system 130. Then, the clearinghouse system 130 performs the steps of the method 301 at block 320 to receive the benefit submission from the dispensary entity processor 140-2. Next, the clearinghouse system 130 performs the steps of the method 301 at block 322 to process the benefit submission and send it to the appropriate entity, which in this case is the system processor(s) 118. Continuing, the system processor(s) 118 performs the steps of the method 301 at block 362 to receive the benefit submission from the clearinghouse system 130 (which had ultimately received the benefit submission from the dispensary entity processor 140-2), and perform a check to see if the benefit submission meets the criteria of the secured program previously opened at block 316, e.g., that the submission has been made within the calendar window opened at block 360. If the criteria matches the secured program, the system processor(s) 118 performs the steps of the method 301 at block 326 to approve the benefit submission. The benefit may then be processed using the appropriate benefit processing business logic based on the benefit specification, as described previously.

If the criteria does not match the secured program, in order to prevent, safeguard against, inhibit or protect against fraud, the system processor(s) 118 performs the steps of the method 301 at block 328 to deny the benefit submission. In such a case, the clearinghouse system 130 performs the steps of the method 301 at block 330 to process the denial, and the dispensary entity processor 140-2 processes the steps of the method 301 at block 332 to receive the denial.

FIGS. 4-5 are flowcharts depicting business logic for execution by the benefit management system of FIG. 1A, in accordance with one or more aspects set forth herein. For example, the business logic examples of FIGS. 4-5 may be provisioned within the benefit processing system based on receiving a provisioning request from a product related entity. These examples in no way limit the scope of the present disclosure, and numerous other business logic examples may be supported by the system.

Turning to FIG. 4, depicted is a business logic 400 that includes numerous processing flows which may occur in different orders. Depending on the order in which the business logic 400 executes, different outcomes result.

In a first example, the business logic 400 may process a benefit submission by executing sequentially blocks 402, 404, 406, 408, and 410. In such a case, at block 402, a pharmacy begins a benefit submission transaction. At block 404, primary insurance coverage is determined. If full or partial coverage is determined, at block 406 secondary billing to a program is determined. If yes, at block 408 a copy assistance program is accessed. Then, at block 410, an amount to pay by the copay assistance program is computed. The business logic 400 then generates a benefit transaction record related to the benefit submission of this example.

In a second example, the business logic 400 may process a benefit submission by executing sequentially blocks 402, 404, 412, 414, and 416. In such a case, at block 402, a pharmacy begins a benefit submission transaction. At block 404, primary insurance coverage is determined. If no coverage is determined, at block 412 secondary billing to a program is determined. If yes, at block 414 a copy assistance program is accessed. Then, at block 416, an amount to pay by the copay assistance program is computed. The business logic 400 then generates a benefit transaction record related to the benefit submission of this example.

In a third example, the business logic 400 may process a benefit submission by executing sequentially blocks 402, 404, 406, 420, 422, 424, and 426. In such a case, at block 402, a pharmacy begins a benefit submission transaction. At block 404, primary insurance coverage is determined. If full or partial coverage is determined, at block 406 secondary billing to a program is determined. If not, at block 420, a manufacturer's coupon is accessed. At block 422, a determination is made of whether there is still a remaining amount to be billed. If yes, at block 424 a copy assistance program is accessed. Then, at block 426, an amount to pay by the copay assistance program is computed. The business logic 400 then generates a benefit transaction record related to the benefit submission of this example.

In a fourth example, the business logic 400 may process a benefit submission by executing sequentially blocks 402, 404, 412, 420, 422, 424 and 426. In such a case, at block 402, a pharmacy begins a benefit submission transaction. At block 404, primary insurance coverage is determined. If no coverage is determined, at block 412 secondary billing to a program is determined. If not, at block 420, a manufacturer's coupon is accessed. At block 422, a determination is made of whether there is still a remaining amount to be billed. If yes, at block 424 a copy assistance program is accessed. Then, at block 426, an amount to pay by the copay assistance program is computed. The business logic 400 then generates a benefit transaction record related to the benefit submission of this example.

FIG. 5 depicts yet other examples of business logic 500-1, 500-2, 500-3 and 500-4. A first example handles a scenario in which primary insurance covers medication but leaves a patient with a co-payment. In such a case, business logic 500-1 at block 502 receives a benefit submission transaction including a claim for payment of a prescription. Then, at block 504 primary insurance partially covers the claim. Next, at block 506, a copay assistance program (one example of which is the National Foundation for Prescription Copay Assistance, or NFPCA) processes and pays as a secondary.

A second example handles a scenario in which primary insurance does not cover medication. In such a case, business logic 500-2 at block 502 receives a benefit submission transaction including a claim for payment of a prescription. Then, at block 505 primary insurance does not cover the claim. Next, at block 506, a copay assistance program (one example of which is the National Foundation for Prescription Copay Assistance, or NFPCA) processes and pays as a secondary.

A third example handles a scenario in which primary insurance covers medication and leaves a patient with a copayment, and secondary covers part of the cost, leaving a remainder copayment. In such a case, business logic 500-1 at block 502 receives a benefit submission transaction including a claim for payment of a prescription. Then, at block 504 primary insurance partially covers the claim. Next, at block 507, a manufacturer coupon is available to cover as a secondary. Next, at block 508, a copay assistance program (one example of which is the National Foundation for Prescription Copay Assistance, or NFPCA) processes and pays as a tertiary.

A fourth example handles a scenario in which primary insurance does not cover medication, the pharmacy bills a manufacturer coupon as a secondary, and the pharmacy bills a tertiary program to cover the remainder of the copay. In such a case, business logic 500-2 at block 502 receives a benefit submission transaction including a claim for payment of a prescription. Then, at block 505 primary insurance does not cover the claim. Next, at block 507, a manufacturer coupon is available to cover as a secondary. Next, at block 508, a copay assistance program (one example of which is the National Foundation for Prescription Copay Assistance, or NFPCA) processes and pays as a tertiary.

The example business logic described above may be encoded in configuration files and loaded by the different product related entities as new programs are added and changed.

FIG. 6 is a block diagram of a computer system 10, such as that employed by the benefit management system 110 of FIG. 1A. A computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed environments where tasks are performed by remote processing devices that are linked through a communications network. In a distributed environment, program modules may be located in both local and remote computer system storage media including memory storage devices.

Computer system/server 12 in computer system 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

Embodiments may include a system, a method, and/or a computer program product. The computer program product may include a computer readable storage medium (or media) having computer readable program instructions thereon for causing a processor to carry out aspects of set forth herein.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the certain embodiments may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects set forth herein.

Embodiments are described herein with reference to flowchart illustrations and/or block diagrams of methods, apparatus (systems), and computer program products according to embodiments. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A medical benefit fraud protection system comprising:
one or more data storage devices storing a plurality of instructions configured to be executed to:
receive a provisioning request from a first processor controlled by a product related entity, the provisioning request comprising a medical benefit specification from the product related entity, the medical benefit specification comprising a plurality of limitations of use;
configure a benefit processing business logic with the provisioning request, the provisioning occurring during continued execution of the instructions;
receive a benefit submission from a second processor controlled by a dispensary entity, the benefit submission relating to the medical benefit specification;
determine an approval decision or a denial decision of the benefit submission based on the limitations of use of the medical benefit specification;
in response to determining an approval decision of the benefit submission, process the benefit submission using the benefit processing business logic based on the benefit specification;
in response to determining a denial decision of the benefit submission, deny the benefit submission;
generate a benefit transaction record or a denial record related to the benefit submission; and
display a first access interface to the product related entity and a second access interface to the dispensing entity, the displaying occurring during the execution of the instructions, wherein the first and second access interfaces are accessible during the execution and comprise different views to different portions of the benefit transaction record related to the benefit submission.

2. The medical benefit fraud protection system of claim 1, wherein the limitations of use in the benefit specification limit the benefit specification to one or more patients.

3. The medical benefit fraud protection system of claim 1, wherein the limitations of use in the benefit specification limit the benefit specification to a period of time.

4. The medical benefit fraud protection system of claim 1, wherein the limitations of use in the benefit specification limit the benefit specification to a number of uses.

5. The medical benefit fraud protection system of claim 1, wherein the limitations of use in the benefit specification limit the benefit specification to one or more patients for a period of time and a number of uses.

6. The medical benefit fraud protection system of claim 1, wherein provisioning the benefit processing business logic comprises generating one or more business rules based on the provisioning request.

7. The medical benefit fraud protection system of claim 1, wherein the product related entity comprises a manufacturer of the medical benefit specification.

8. The medical benefit fraud protection system of claim 1, wherein the benefit submission is received via a clearinghouse.

9. The medical benefit fraud protection system of claim 1, wherein the benefit submission is not received via a clearinghouse.

10. The medical benefit fraud protection system of claim 1, wherein the plurality of instructions are further configured to be executed to receive a plurality of provisioning requests from a plurality of product related entities, the plurality of provisioning requests comprising specifications of a plurality of benefit specifications from the plurality of product related entities.

11. The medical benefit fraud protection system of claim 10, wherein the plurality of instructions are further configured to be executed to provision the benefit processing business logic of the medical benefit fraud protection system with the plurality of provisioning requests, the provisioning occurring during continued operation of the medical benefit fraud protection system.

12. A medical benefit fraud protection method, the method comprising:
receiving a provisioning request from a first processor controlled by a product related entity, the provisioning request comprising a benefit specification from the product related entity, the benefit specification comprising a plurality of limitations of use;
configuring a benefit processing business logic with the provisioning request;
receiving a benefit submission from a second processor controlled by a dispensary entity, the benefit submission relating to the benefit specification from the product related entity;
determining an approval decision or a denial decision of the benefit submission based on the limitations of use of the benefit specification;
in response to determining an approval decision of the benefit submission, processing the benefit submission using the benefit processing business logic based on the benefit specification;
in response to determining a denial decision of the benefit submission, denying the benefit submission;
generating a benefit transaction record related to the benefit submission; and
displaying a first access interface to the product related entity and a second access interface to the dispensing entity, the displaying occurring during execution, wherein the first and second access interfaces are accessible and comprise different views to different portions of the benefit transaction record related to the benefit submission.

13. The medical benefit fraud protection method of claim 12, wherein the limitations of use in the benefit specification limit the benefit specification to one or more patients.

14. The medical benefit fraud protection method of claim 12, wherein the limitations of use in the benefit specification limit the benefit specification to a period of time.

15. The medical benefit fraud protection method of claim 12, wherein the limitations of use in the benefit specification limit the benefit specification to a number of uses.

16. The medical benefit fraud protection method of claim 12, wherein the limitations of use in the benefit specification limit the benefit specification to one or more patients for a period of time and a number of uses.

17. The medical benefit fraud protection method of claim 12, wherein provisioning the benefit processing business logic comprises generating one or more business rules based on the provisioning request.

18. A medical benefit fraud protection system comprising:
one or more data storage devices storing a plurality of computer-readable instructions configured to be executed to:
receive a provisioning request from a product related entity, the provisioning request comprising a medical benefit specification from the product related entity, the medical benefit specification comprising a plurality of limitations of use associated with a product marketed by the product related entity;
receive a benefit submission from a medical merchant, the benefit submission relating to the medical benefit specification;
determine whether the benefit submission is approved or denied based on the limitations of use;
in response to the determination of approval, process the benefit submission, generate a benefit transaction record, and cause the benefit transaction record to be graphically indicated; and
in response to the determination of denial, deny the benefit submission and generate a benefit denial record, and cause the benefit denial record to be graphically indicated.

19. The medical benefit fraud protection system of claim 18, wherein the limitations of use of the benefit specification limit the benefit specification to a unique identity of a customer.

20. The medical benefit fraud protection system of claim 18, wherein the limitations of use of the benefit specification limit the benefit specification to a designated period of time.
